(19) United States
(12) Patent Application Publication
(10) Pub. No.: US 2008/0115890 A1
(43) Pub. Date: May 22, 2008

(54) Methods and systems for using molecular-film adhesives

(76) Inventors: Sean X. Zhang, Sunnyvale, CA (US); Patricia A. Beck, Palo Alto, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/519,544

(22) Filed: Sep. 11, 2006

(51) Int. Cl. C09J 4/00; C07D 213/04

(52) U.S. Cl. 156/336; 156/60; 156/326; 546/340

(57) ABSTRACT

Various embodiments of the present invention are directed to molecular-film adhesives and methods and systems for using molecular-film adhesives. In one embodiment of the present invention, an amphipathic, biological-substrate-compatible adhesive is applied as a molecular-film. The amphipathic adhesive includes a first functional group, capable of bonding to a first substrate, and a second functional group, capable of bonding to a second, chemically dissimilar substrate.

### TECHNICAL FIELD

The present invention relates to adhesives, and, in particular, to amphipathic adhesives applied as a molecular-film for binding heterogeneous substrates.

### BACKGROUND OF THE INVENTION

Typical adhesives used in household applications, such as white glue or rubber cement, are classified as drying adhesives. Drying adhesives are typically composed of polymers dissolved in a solvent, or monomers that chemically react to form polymers. As the solvent evaporates, the adhesive hardens and binds materials. The chemical composition of most drying adhesives determines the degree to which the drying adhesive adheres to different materials. However, drying adhesives typically exhibit weak adhesive properties, particularly with respect to non-porous substrates, and may not exhibit sufficiently diverse adhesive characteristics for bonding together chemically dissimilar surfaces (“heterogeneous substrates”). In addition, drying adhesives may form voids between substrates, allow for movement of the substrates upon application of the adhesive, produce runoff from bonded-substrate interfaces, and may need long curing times.

Reactive adhesives operate by chemically bonding with substrate molecules. Traditional examples include two-part epoxy, peroxide, silane, metallic cross-linking agents, and isocyanate. However, reactive adhesives are typically suitable only for bonding together chemically similar surfaces (“homogeneous substrates”). For example, molecules serving as adhesives typically have an affinity for only one type of substrate, because only one type of functional group of the adhesive molecule chemically bonds with substrate molecules.

Binding metallic or synthetic-polymer surfaces to biological materials, including biological membranes (“bio-membranes”), and skin, is one example of many new, challenging applications for adhesives. Not only are biological materials complex and highly hydrated, they are also easily damaged by synthetic, reactive adhesives. With the advent of new sensors, drug delivery devices, biological analytical devices, and other devices, efforts are currently directed to development of adhesives for binding non-biological materials to biological substrates. Researchers, manufacturers, and consumers have recognized a need for adhesives that can be used to bind together heterogeneous substrates, including biological substrates.

### SUMMARY OF THE INVENTION

Various embodiments of the present invention are directed to molecular-film adhesives and methods and systems for using molecular-film adhesives. In one embodiment of the present invention, an amphipathic, biological-substrate-compatible adhesive is applied as a molecular-film. The amphipathic adhesive includes a first functional group, capable of bonding to a first substrate, and a second functional group, capable of bonding to a second, chemically dissimilar substrate.

### BRIEF DESCRIPTION OF THE DRAWINGS

FIG. 1 shows two heterogeneous substrates with chemically dissimilar surfaces.

FIG. 2 shows an amphipathic adhesive molecule that represents an embodiment of the present invention.

FIG. 3 shows two heterogeneous substrates bonded together by the amphipathic adhesive molecules according to an embodiment of the present invention.

FIG. 4 shows chemical structures for three different amphipathic adhesive molecules that represent embodiments of the present invention.

FIGS. 5A-B show amphipathic adhesive molecules bonded to a micro-needle array according to an embodiment of the present invention.

FIGS. 6A-E show bonding of a micro-needle array to two substrates by an amphipathic adhesive according to an embodiment of the present invention.

FIG. 7 is a control-flow diagram illustrating a method of using a molecular-film adhesive that represents an embodiment of the present invention.

### DETAILED DESCRIPTION OF THE INVENTION

Various embodiments of the present invention are directed to molecular-film adhesives and methods and systems for using molecular-film adhesives. Embodiments of the present invention include amphipathic adhesives applied to form monolayers, or molecular films on a first substrate, binding to the first substrate via a first functional group. Molecules of the amphipathic adhesive include a second functional group with low affinity for the first substrate, but which binds with high affinity to a second substrate. The two chemically dissimilar functional groups allow the amphipathic adhesive substance to bind together two chemically dissimilar surfaces.

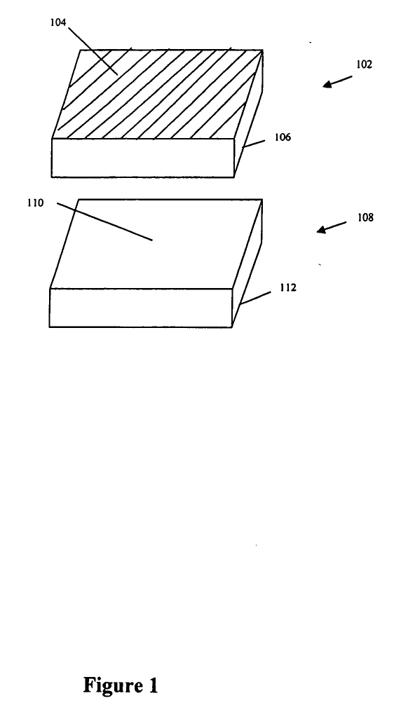

FIG. 1 shows two heterogeneous substrates with chemically dissimilar surfaces. The bottom surface 106 of the first substrate 102 is oriented directly above the top surface 110 of the second substrate 108. The first substrate 102 can be a metal, a synthetic substance, such as a polymeric material or a ceramic, and may be the surface of a sensor, drug-delivery device, or analytical device. The second substrate can be any material with a surface chemically different from the surface of the first substrate, including a biological assay chip (“bioassay chip”), another biological analytical device, a bio-membrane, skin, or another type of natural or artificial substrate.

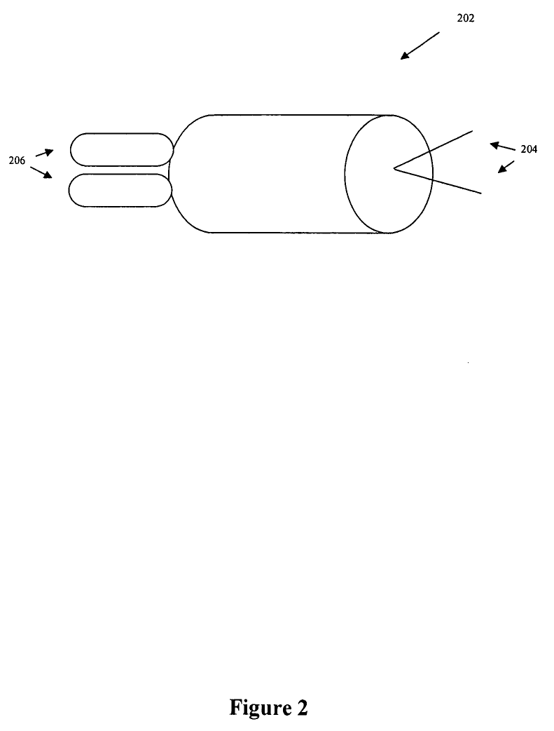

FIG. 2 shows an amphipathic adhesive molecule that represents an embodiment of the present invention. The amphipathic adhesive molecule includes at least two functional groups with different affinities for external functional groups or substrates. The first and second functional groups 204 and 206 of the amphipathic adhesive molecule facilitate two different characteristic chemical reactions in which the molecule participates. The first functional group may be, for example, an amino, thiol, hydroxyl, pyridyl, imidazolyl or an anhydride group, and may therefore exhibit an affinity for electrophilic substrates, such as metals, while the second functional group may be, for example, carboxyl, phosphate, aldehyde, ketone, hydroxyl, or a functional group capable of reacting with free amines, and may therefore exhibit affinity for nucleophilic substrates, including certain functional groups of proteins. In the example of FIGS. 1-3, the first functional group 204 reacts with surface molecules of the first substrate 102, described above with reference to FIG. 1, to form a covalent bond or a coordination bond, and the second functional group 206 reacts with surface molecules of the second substrate 108, described above with reference to FIG. 1, to form a strong ionic, covalent, or other type of bond.

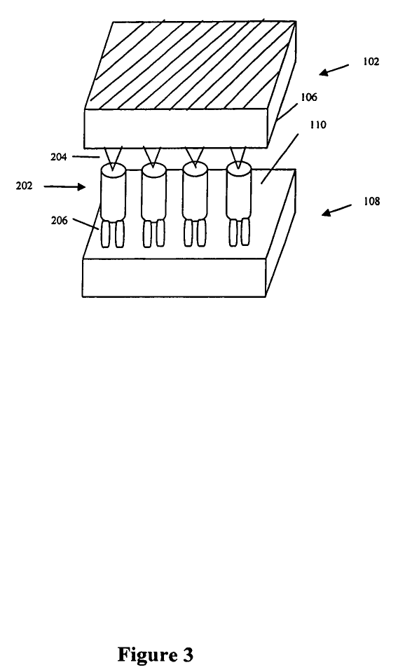

FIG. 3 shows two heterogeneous substrates bonded together by the amphipathic adhesive molecules according to an embodiment of the present invention. The amphipathic adhesive molecules 202 bind the bottom surface 106 of the first substrate 102 through the first functional groups 204, and bind the top surface 110 of the second substrate 108 through the second functional groups 206. The amphipathic adhesive is applied to one substrate to form a molecular film, which is dimensionally and positionally stable, in contrast to typical adhesives. The molecular film may be applied in a single mono-layer or as multiple mono-layers.

When bonding biological materials, the amphipathic adhesive can be selected to be non-irritating and to have strong adhesion to hydrated surfaces. In addition, a multi-step process may be used to facilitate binding both to a biological substrate and a synthetic or metallic substrate. For example, the pH may be lowered when bonding to the first substrate 102, and the pH then raised for bonding to the second substrate 108, such as a biological substrate.

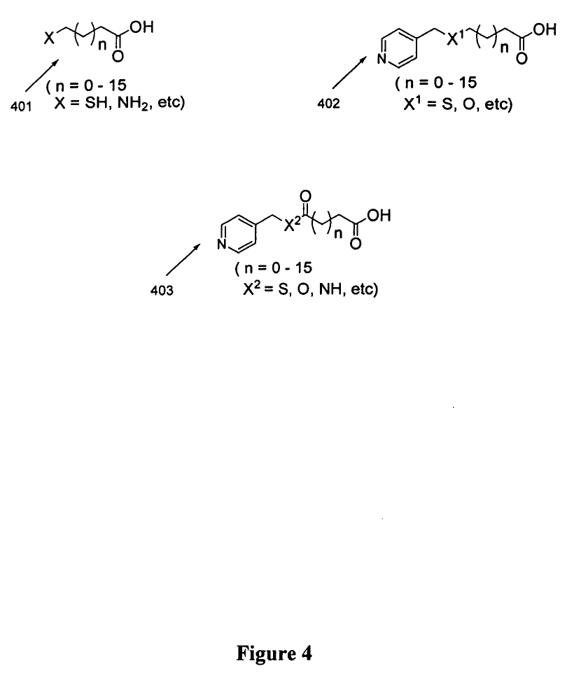

FIG. 4 shows chemical structures for three different amphipathic adhesive molecules that represent embodiments of the present invention. The chemical structures shown in FIG. 4 include: a substituted carboxylic acid including one of a thiol or amine substituent 401, a substituted carboxylic acid including a pyridyl substitutent and one of a ether or thio-ether substitutent 402, and a substituted carboxylic acid including a pyridyl substitutent and one of an amide or ester substitutent 403. In additional embodiments of the present invention, an amino acid, lysine, cystein, cystine, and an amine-substituted carbohydrate may be employed. Additional embodiments of the present invention include derivatives of a substituted carboxylic acid including one of a thiol or an amine substituent, a substituted carboxylic acid including a pyridyl substitutent and one of a ether or a thio ether substitutent, and a substituted carboxylic acid including a pyridyl substitutent and one of an amide or a ester substitutent, an amino acid, lysine, cystein, cystine, and an amine-substituted carbohydrate.

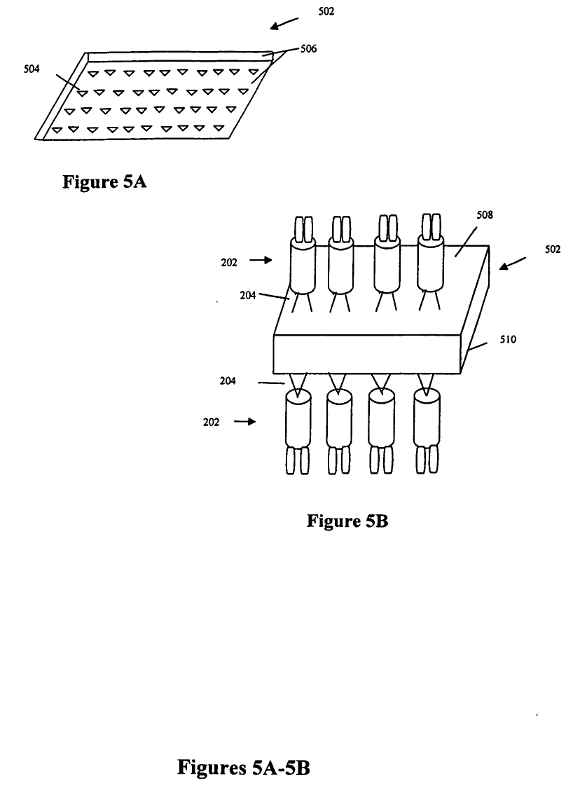

FIGS. 5A-B show amphipathic adhesive molecules bonded to a micro-needle array according to an embodiment of the present invention. The micro-needle array comprises a thin layer of metal, or a conductive polymer, in which a regular array of micro-needles 504 is formed. An amphipathic adhesive is applied as a molecular film 506 to the exposed surfaces of the micro-needle array 502. The molecular film of amphipathic adhesive may be applied in either the liquid or gas phase. The amphipathic adhesive molecules bind to the exposed surfaces of the micro-needle array including, the top and bottom surfaces 508 and 510 of the micro-needle array 502, as shown in FIG. 5B, through the first functional group 204 of the amphipathic adhesive molecule.

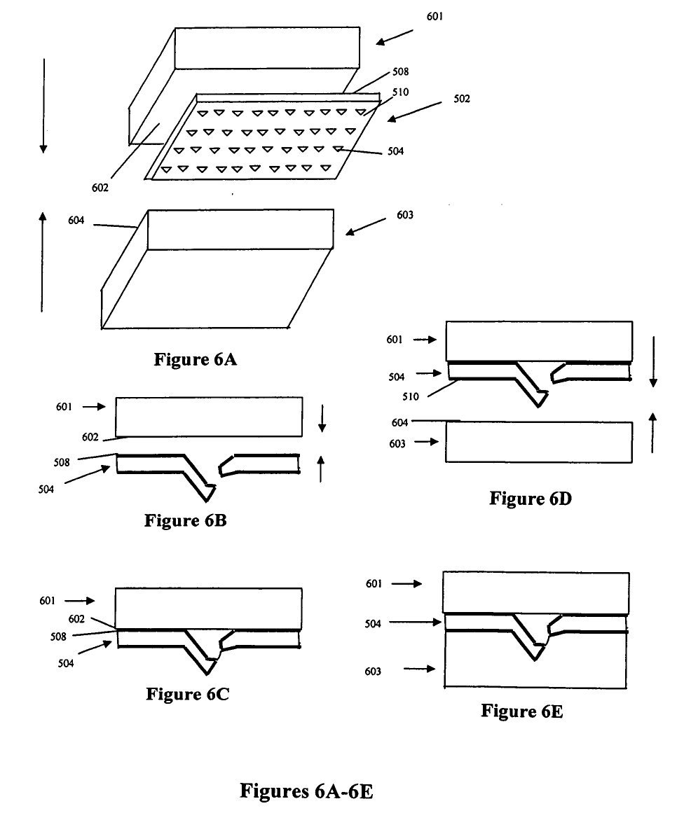

FIGS. 6A-E show bonding of a micro-needle array to two substrates by an amphipathic adhesive according to an embodiment of the present invention. First, as shown in FIG. 6A, two substrates 601 and 603, and a micro-needle array 502 are provided. Next, as shown in FIG. 6B, an amphipathic adhesive is applied to either a top surface 508 of the micro-needle array, to a bottom surface 602 of a device substrate 601, or to both the top surface of the micro-needle array and the bottom surface of the device substrate. The micro-needle array 502 and the device substrate 601 are brought into contact. The micro-needle array is then bonded to the bottom surface of the device substrate 601, such as a bioassay chip, to form a biosensor, as shown in FIG. 6C. Next, as shown in FIG. 6D, a second application of the same amphipathic adhesive or a different amphipathic adhesive is applied either to a bottom surface 510 of the micro-needle array, a top surface 604 of a second substrate, or to both the bottom surface of the micro-needle array and the top surface of the second substrate. The biosensor, or micro-needle array bonded to the device substrate, and the second substrate 603 are brought into contact. The biosensor is then bonded to the top surface 604 of the second substrate 603, such as skin, the micro-needle 504, in one embodiment of the present invention, penetrating the skin, embedding the biosensor into the skin to allow for biological analysis, as shown in FIG. 6E. In an alternative embodiment of the present invention, the biosensor may be embedded into the skin by the micro-needle penetrating the skin, without a second application of the same amphipathic adhesive or a different amphipathic adhesive. In an alternative embodiment of the present invention, a second application of the same amphipathic adhesive or a different amphipathic adhesive is applied to the bottom surface 510 of the micro-needle array without application to tips of the micro-needles 504. In various alternative embodiments of the present invention, the amphipathic adhesive may be applied in specific locations, to specific features, or, by contrast, to an entire surface. Of course, the micro-needle could be first bonded to the skin, and the biosensor could then be bonded to the micro-needle. The micro-needle array 502 serves as the first substrate 102, in the example of FIGS. 1-3. The device substrate 601 and the second substrate 603 are each heterogeneous to the micro-needle array, both representing the second substrate in the example of FIGS. 1-3. A large number of device substrates may be used, including a bioassay chip, a drug delivery device, and fluid extraction device.

Advantages of a molecular-film adhesive include dimensional and positional stability, biological affinity, extremely low-volume application which prevents, in the above example, clogging of micro-needles, and the ability to tailor an amphipathic adhesive to better adhere with each substrate of a substrate pair. In an alternative embodiment of the present invention, the amphipathic adhesive may be used to provide a non-irritating, temporary bond to the skin. Wound care dressings, athletic tape, analgesic and transdermal drug patches may be bound to skin and membranous tissue using an amphipathic adhesive. Applications related to skin grafts for burn victims and body art are also possible.

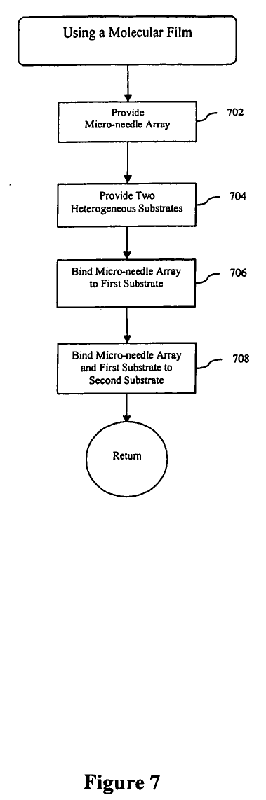

FIG. 7 is a control-flow diagram illustrating a method of using a molecular-film adhesive, that represents an embodiment of the present invention, to fabricate and apply a micro-needle based device. In step 702, a micro-needle array is provided, as described above in the description of FIGS. 5A-5B. In step 704, two heterogeneous substrates are provided, as described above in the description of FIG. 1. In step 706, the micro-needle array is bonded, using a first molecular-film adhesive, as described above in the description of FIG. 6A. In step 708, the micro-needle array and first substrate are bound to the second substrate, as described above in the description of FIG. 6A, using the same or a different molecular-film adhesive.

Additional modifications within the spirit of the invention will be apparent to those skilled in the art. In an alternative embodiment of the present invention, an amphipathic adhesive may be used to bond a solid micro-needle, used as a probe to sense electrical signals or to apply stimulation electrical signals, to a heterogeneous substrate. A micro-needle may be covered with a variety of materials through processes such as plating to enhance or modify structural, chemical, or biological properties. In an alternative embodiment of the present invention, other microelectromechanical system (“MEMS”) devices and microparts may be used. Lab-on-a-chip (“LOC”) devices which scale down single or multiple lab processes to a chip-format fall into this category. LOC use may include analysis such as chemical analysis, environmental monitoring and medical diagnostics, and also synthetic chemistry such as rapid screening and microreactors for pharmaceutics. One of the basic building blocks of MEMS device production, is the ability to deposit thin films of material, such as a molecular film of adhesive. In addition, electrodes, such as electrocardiograph (“EKG”) electrodes may be placed on the skin with less irritation, and less lost of electrical signal than with conventional adhesives and backings. Use of an amphipathic adhesive in optical systems would result in less loss of an optical signal than with conventional adhesives. A molecular-film adhesive is any adhesive that can be applied to form a mono-layer, an oriented mono-layer, nanometer-scale layer, submicron-scale layer, or physically or chemically linked multiple layers (“multilayers”). Multilayers may be bonded by any of a large number of bond types, including ionic bonding, chelated-metal-complex bonding, and covalent bonding. Mono-layers or other layers may vary in composition, molecular organization and orientation, and in other ways. In various alternative embodiments of the present invention, the amphipathic adhesive may be applied to specific locations on the surface of an object, to specific features of an object, or, by contrast, to the entire surface of an object, as needed for a given application.

The foregoing detailed description, for purposes of illustration, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Obviously many modifications and variation are possible in view of the above teachings. The embodiments were chosen and described in order to best explain the principles of the invention and its practical applications and to thereby enable others skilled in the art to best utilize the invention and various embodiments with various modifications as are suited to the particular use contemplated.

1. An amphipathic adhesive comprising:
amphipathic molecules, each having a first functional group capable of bonding to a first substrate, and a second functional group capable of bonding to a second, dissimilar substrate;
the amphipathic adhesive applied to the first substrate to form a molecular film to which the second substrate subsequently binds.

2. The amphipathic adhesive of claim 1 wherein the molecular film is one of:
a mono-layer;
an oriented mono-layer;
a nano-scale layer;
a submicron-scale layer;
a physically linked multilayer; and
a chemically linked multilayer.

3. The adhesive of claim I wherein the first functional group further comprises one of:
an amino group;
a thiol group;
a hydroxyl group;
a pyridyl group;
an imidazolyl group; and
an anhydride group.

4. The adhesive of claim I wherein the second functional group further comprises one of:
a carboxyl group;
a phosphate group;
an aldehyde group;
a ketone group;
a hydroxyl group; and
a functional group capable of reacting with free amines.

5. The adhesive of claim 1 wherein the first substrate further comprises one of:
a metal;
a conductive polymer;
a synthetic substance;
a sensor;
a drug delivery device; and
an analytical device.

6. The adhesive of claim 6 wherein the synthetic substance further comprises one of:
a polymeric material; and
a ceramic.

7. The adhesive of claim 1 wherein the second substrate further comprises one of:
a bioassay chip;
a biological analytical device;
a bio-membrane;
a skin;
a natural substrate; and
an artificial substrate.

8. The adhesive of claim 1 wherein the amphipathic adhesive further comprises one of:
a substituted carboxylic acid including one of a thiol or amine substitutent;
a substituted carboxylic acid including a pyridyl substitutent and one of a ether or thio-ether substitutent;
a substituted carboxylic acid including a pyridyl substitutent and one of an amide or ester substitutent;
an amino acid;
lysine;
cystein;
cystine; and
an amine-substituted carbohydrate.

9. The adhesive of claim 8 wherein the amphipathic adhesive further comprises a chemical derivative of one of:
a substituted carboxylic acid including one of a thiol or amine substitutent;
a substituted carboxylic acid including a pyridyl substitutent and one of a ether or thio-ether substitutent;
a substituted carboxylic acid including a pyridyl substitutent and one of an amide or ester substitutent;
an amino acid;
lysine;
cystein;
cystine; and
an amine-substituted carbohydrate.

10. A method for fabricating a biological analytical device and affixing the biological analytical device to a biological substrate, the method comprising:
bonding an analytical device to a micro-needle array to produce a biological analytical device; and
binding the biological analytical device to skin or tissue using a molecular-film adhesive.

11. The method of fabricating of claim 10 wherein bonding the analytical device to the micro-needle further includes applying a molecular-film adhesive to the micro-needle array.

12. The method of fabricating of claim 10 wherein the micro-needle array further comprises one of:
a metal;
a conductive polymer;
a synthetic substance;
a sensor;
a drug delivery device; and
an analytical device.

13. The method of fabricating of claim 10 wherein the molecular-film adhesive further comprises one of:
a substituted carboxylic acid including one of a thiol or amine substitutent;
a substituted carboxylic acid including a pyridyl substitutent and one of a ether or thio-ether substitutent;
a substituted carboxylic acid including a pyridyl substitutent and one of an amide or ester substitutent;
an amino acid;
lysine;
cystein;
cystine; and
an amine-substituted carbohydrate.

14. The method of fabricating of claim 13 wherein the molecular-film adhesive further comprises a chemical derivative of one of:
a substituted carboxylic acid including one of a thiol or amine substitutent;
a substituted carboxylic acid including a pyridyl substitutent and one of a ether or thio-ether substitutent;
a substituted carboxylic acid including a pyridyl substitutent and one of an amide or ester substitutent;
an amino acid;
lysine;
cystein;
cystine; and
an amine-substituted carbohydrate.
